(19) United States
(12) Patent Application Publication
(10) Pub. No.: US 2026/0153489 A1
(43) Pub. Date: Jun. 4, 2026

(54) TRACEABLE CALIBRATION DEVICE AND METHOD OF PARTILE NUMBER ANALYZERS FOR VEHICLE EMISSION

(71) Applicant: national institute of metrology, China, Beijing (CN)

(72) Inventors: Junjie Liu, Beijing (CN); Ji Xiao, Beijing (CN)

(21) Appl. No.: 19/372,097

(22) Filed: Oct. 28, 2025

(30) Foreign Application Priority Data
Dec. 3, 2024 (CN) 202411755746.3

(51) Int. Cl. G01N 33/00; G01N 15/06

(52) U.S. Cl. CPC G01N 33/0006; G01N 15/0656

(57) ABSTRACT

A traceable calibration device of particle number (PN) analyzers for vehicle emission includes a particle generation system, used for generating different types of aerosol samples; a solenoid valve system, used for controlling various aerosol samples to quantitatively enter the particle growth system; a particle growth system, used for performing charge classification to obtain doubly-charged-particle aerosol samples, triply-charged-particle aerosol samples and monodisperse aerosol samples, and obtaining grown particle samples by controlling the growth speed of the monodisperse aerosol samples through temperature adjustment; and a particle measurement system, used for measuring electrical current and PN concentration of the grown particle samples. In the present disclosure, the problem of low calibration accuracy of a PN analyzer for vehicle emission in the related art is solved.

### CROSS-REFERENCE TO RELATED APPLICATION

This application claims priority of Chinese Patent Application No. 202411755746.3, filed on Dec. 3, 2024, the entire contents of which are incorporated herein by reference.

### TECHNICAL FIELD

The present disclosure relates to the technical field of particle number (PN) measurement, and in particular to a traceable calibration device and method of PN analyzers for vehicle emission (hereafter referred to as the PN analyzer).

### BACKGROUND

Vehicle exhaust emission is one of the most important sources of PM2.5. Therefore, improving the control level of vehicle pollutant emission and reducing the pollution emission of vehicles have become one of the main ways and methods to control atmospheric particulate pollutants. In view of the above reasons, various automobile manufacturers and automobile testing institutions have purchased PN analyzers for vehicle emission and established corresponding testing capabilities, which are used for PN and concentration emission performance evaluation of engines and complete vehicles and diesel particulate filter (DPF) filtration performance evaluation of PM capture systems, etc., providing technical guidance for technical upgrading of vehicles and accessories.

The PN analyzer is a relatively complex measuring system, including a condensation particle counter (CPC), a particle sampling probe (PSP), a particle transmission tube (PTT), a pre-classifier fractionator (PCF), a volatile particle remover (VPR), etc. According to the working mode, the PN analyzer can be divided into two types: stationary type and portable type. In order to ensure the reliability of the measurement data of the PN analyzer, the measurement performance requirements for the PN analyzer used is clearly stipulated in the National standards for vehicle emission (China VI), such as: counting efficiency in different particle size ranges, particle concentration attenuation coefficient, measurement linearity, removal efficiency of volatile particles, accuracy of dilution ratio, etc. However, due to the lack of corresponding measurement standards in China, the above core measurement performance cannot be calibrated and effectively confirmed.

At present, the calibration of PN analyzer for vehicle emission is completed by comparing the instrument to be calibrated with the standard instrument. Standard instruments are typically calibrated aerosol electrometers or CPCs. However, in the method of comparison calibration, there are the following technical problems. 1. The calibrated upper limit of concentration of standard instruments usually does not exceed 20,000 particles/cm³, to calibrate the high concentration range of PN analyzers, it is necessary to connect the aerosol diluter at the entrance of the standard instrument to ensure that the measurement results of standard instruments are within the calibration range. However, due to the introduction of calibration uncertainty of aerosol diluter, the uncertainty of the final calibration result increases accordingly. 2. It is difficult to produce aerosol samples with high concentration and large particle size by using conventional methods, making it difficult to effectively evaluate the indication error of PN analyzers for particles above 100 nm. 3. When using the aerosol electrometer to calibrate the PN analyzers, the presence of multi-charged particles in the test aerosol will affect the calibration results. 4. The mismatch between the flow rates of the standard instrument and the calibrated instrument will lead to the deviation of the calibration results. 5. In PN analyzer calibration, commonly used particulate materials typically include polystyrene, silver, soot, NaCl, etc. However, due to the differences in composition, particle size and other characteristics between particulate materials from vehicle and calibration aerosol materials, certain discrepancies may exist for calibration results by using those different materials.

### SUMMARY

In order to address the limitations of existing technologies, an objective of the present disclosure is to provide a traceable calibration device and method of PN analyzers for vehicle emission to solve the existing issue of low calibration accuracy of PN analyzer for vehicle emission.

In order to achieve the above objective, the present disclosure adopts the following technical solutions.

The present disclosure provides a traceable calibration device of PN analyzers for vehicle emission, including:
a particle generation system, a solenoid valve system, a particle growth system and a particle measurement system connected in sequence; and
the particle generation system is used for producing different types of aerosol samples; the solenoid valve system is used for controlling and quantitatively delivering various aerosol samples to the particle growth system; the particle growth system is used for classifying different types of aerosol samples to obtain monodisperse aerosol samples, doubly-charged-particle aerosol samples and triply-charged-particle aerosol samples, determining concentrations of the three aerosol samples, and obtaining grown particle samples by controlling the growth speed of the monodisperse aerosol samples through temperature adjustment; the particle measurement system is used for measuring the particle number of grown particle samples, or the electrical current derived by the particle. The solenoid valve system is set as seven electromagnetic valves, and the different types of solid aerosol samples include polystyrene, NaCl, silver (Ag) and soot.

Preferably, the particle generation system includes:
a pneumatic atomization generator, a diffusion dryer, an evaporation condensation generator and a soot generator which are all connected to the solenoid valve system;
the pneumatic atomization generator is connected to the diffusion dryer; and
the pneumatic atomization generator is used for atomizing polystyrene suspension into micron-sized droplets; the diffusion dryer is used for evaporating the micron-sized droplets to obtain a solid polystyrene aerosol sample; the evaporation condensation generator is used for generating NaCl and Ag solid aerosol samples, and the soot generator is used for generating soot aerosol samples.

Preferably, the particle growth system includes:
an aerosol neutralizer, a first differential electrical mobility classifier (DEMC), a second DEMC, a third DEMC, a CPC, a T-valve, a temperature controller, and a particle grower;
the aerosol neutralizer is connected to the solenoid valve system, the aerosol neutralizer is connected to the first DEMC, the second DEMC and the third DEMC, respectively, the CPC is connected to the second DEMC, the third DEMC and the T-shaped valve, respectively. The first DEMC is connected to the T-shaped valve, and the particle grower is connected to the T-shaped valve and the temperature controller; and
the aerosol neutralizer is used for neutralizing the charges of different types of aerosol samples to obtain overall electrically neutral aerosol sample; the first DEMC is used for classifying the aerosol sample to obtain monodisperse aerosol sample; the second DEMC is used for classifying the aerosol sample to obtain aerosol sample with two charged particles; the third DEMC is used for classifying the aerosol sample to obtain aerosol sample with three charged particles; the CPC is used for measuring concentrations of the monodisperse aerosol sample, the aerosol sample with two charged particles and the aerosol sample with three charged particles; the T-shaped valve is used for controlling the monodisperse aerosol samples to enter a particle measurement system; the temperature controller is used for controlling the temperature of the particle grower; and the particle grower is used for controlling the growth speed of the monodisperse aerosol samples according to the temperature.

Preferably, the particle measurement system includes:
a pressure gauge, a mixing and sampling tube, a particle size spectrometer, an aerosol electrometer, a PN analyzer, a constant flow pump, a first mass flowmeter and a second mass flowmeter;
one end of the mixing and sampling tube is connected to the T-shaped valve and the particle grower, the pressure gauge is mounted at a first preset position at a front end of the mixing and sampling tube, and the particle size spectrometer is arranged at a second preset position at the front end of the mixing and sampling tube; the particle number measuring instrument is arranged at a first preset position at a rear end of the mixing and sampling tube, and the aerosol electrometer is arranged at a second preset position at the rear end of the mixing and sampling tube; and the first mass flowmeter and the second mass flowmeter are connected to the constant flow pump, the second mass flowmeter is connected to the other end of the mixing and sampling tube, and the first mass flowmeter is connected to the aerosol electrometer; and
the mixing and sampling tube is used for mixing the monodisperse aerosol samples, the doubly-charged-particle aerosol samples and the triply-charged-particle aerosol samples; the particle size spectrometer is used for real-time monitoring the particle size distribution of aerosol samples; the pressure gauge is used for monitoring the pressure in the mixing and sampling tube; the aerosol electrometer is used for measuring the electrical current and particle concentration of charged particles; the constant flow pump is used for extracting aerosol samples from the mixing and sampling tube and the aerosol electrometer; the first mass flowmeter is used for controlling the measured flow of the aerosol electrometer; and the second mass flowmeter is used for controlling the aerosol flow in the mixing and sampling tube.

A traceable calibration method of PN analyzers for vehicle emission includes the steps of:
turning on the aerosol electrometer and a PN analyzer, adjusting flow rates and keeping the flow rates consistent;
setting the first DEMC, the second DEMC and the third DEMC to correspond to a first preset particle size, a second preset particle size, a third preset particle size, a first preset voltage size, a second preset voltage size and a third preset voltage size, and keeping a sheath gas flow rate and an aerosol flow rate consistent;
controlling the opening and closing of corresponding solenoid valves in the solenoid valve system, obtaining different solid aerosol samples, i.e., polystyrene, NaCl, Ag and soot by using the pneumatic atomization generator, evaporation condensation generator, and soot generator, and transmitting the samples to the first DEMC, the second DEMC and the third DEMC to obtain aerosol samples with different charge states;
using the CPC to measure concentrations of the aerosol samples with different charge states and obtain their fractions, and by using those results, correcting the measurement result of the aerosol electrometer;
causing aerosol samples from the first DEMC to enter the mixing and sampling tube by using the T-shaped valve, and controlling the flow and pressure of the mixing and sampling tube using a mass flow controller and dilution air; and
obtaining particle counting efficiencies for different types of aerosol samples according to the measured values, average values of the aerosol electrometer and the PN analyzer.

Preferably, an expression of the particle counting efficiency is:

$${η}_{PN} = \frac{{\overline{C}}_{PN}}{{\overline{C}}_{F}} \times {η}_{F} \times {\sum }_{p = 1}^{n} ( {φ}_{p} \times p ) \times 100 \%$$

where η_{PN} is a particle counting efficiency of the PN analyzer to be calibrated; C̅_{PN} is an average of measurement results of the PN analyzer to be calibrated, in particles/cm³; C̅_{F} is an average of measurement results of the aerosol electrometer, in particles/cm³; η_{F} is a particle counting efficiency of the aerosol electrometer, which is a dimensionless quantity; φₚ is a fraction of particles carrying p charges in aerosol particles, which is a dimensionless quantity; and p is the number of positive or negative charges carried by particles, which is a dimensionless quantity.

The present disclosure provides the following technical effects.

The present disclosure provides a traceable calibration device and method of PN analyzers for vehicle emission, including a particle generation system, a solenoid valve system, a particle generating system and a particle measurement system connected in sequence. The particle generation system is used for different types of aerosol samples; the solenoid valve system is used for controlling various aerosol samples to quantitatively enter the particle growth system; the particle growth system is used for classifying aerosol samples to obtain doubly-charged-particle aerosol samples, triply-charged-particle aerosol samples and monodisperse aerosol samples, and obtaining grown particle samples by controlling the growth speed of the monodisperse aerosol samples through temperature adjustment; the particle measurement system is used for measuring electrical current and PN concentration of the grown particle samples; and the solenoid valve system is configured with seven electromagnetic valves, and the different types of aerosol samples include polystyrene, NaCl, Ag and soot. In the present disclosure, the counting efficiency of the PN analyzer in the range of (10-200) nm can be calibrated by this method, where, several typical aerosol samples of different materials could be generated, i.e., polystyrene, NaCl, Ag, soot, and by using this method, the influence of aerosol samples of different materials on the particle counting efficiency of the PN analyzer can be effectively evaluated. At the same time, through the combination of evaporation condensation generation technology and particle grower, monodisperse aerosol samples with high concentration (10⁶ particles/cm³) and large particle size (200 nm) can be effectively produced. The used measurement standard, which is an aerosol electrometer with adjustable flow rate, built-in flow calibration curve, multi-charge correction factor and flow correction factor, can output current and PN concentration, and can accurately measure PN concentration in a large range (10⁶ particles/cm³), with results traceable to the international system of units (SI).

### BRIEF DESCRIPTION OF THE DRAWINGS

To explain the technical solutions of examples in the present disclosure or in the related art more clearly, the accompanying drawings required in the description of the examples are introduced briefly below. Obviously, the drawings in the following description are only some examples of the present disclosure, and other drawings can be obtained according to these drawings without creative efforts for those ordinary skilled in the art.

FIG. 1 is a schematic structural diagram of a traceable calibration device of PN analyzers for vehicle emission according to an example of the present disclosure.

### DETAILED DESCRIPTION

Technical solutions in the examples of the present disclosure will be described clearly and completely in the following with reference to the accompanying drawings in the examples of the present disclosure. Obviously, all the described examples are only some, rather than all examples of the present disclosure. Based on the examples in the present disclosure, all other examples obtained by those ordinary skilled in the art without creative efforts belong to the protection scope of the present disclosure.

In order to make the above objectives, features and advantages of the present disclosure more obvious and understandable, the present disclosure is further explained in detail in combination with the accompanying drawings and specific embodiments.

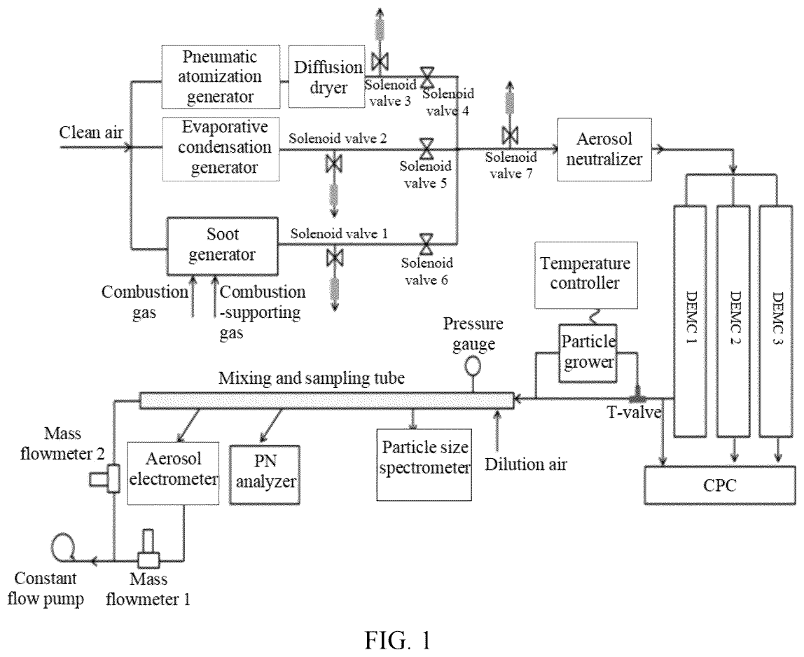

As shown in FIG. 1, the present disclosure provides a traceable calibration device of PN analyzers for vehicle emission, including:
a particle generation system, a solenoid valve system, a particle generating system and a particle measurement system connected in sequence, in which
the particle generation system is used for generating different types of aerosol samples; the solenoid valve system is used for controlling various aerosol samples to quantitatively enter the particle growth system; the particle growth system is used for classifying different types of aerosol samples to obtain doubly-charged-particle aerosol samples, triply-charged-particle aerosol samples and monodisperse aerosol samples, and obtaining grown particle samples by controlling the growth speed of the monodisperse aerosol samples through temperature adjustment; the particle measurement system is used for measuring the grown particle samples to obtain output electrical current and PN concentration; and the solenoid valve system is configured with seven electromagnetic valves, and the different types of solid aerosol samples include polystyrene, NaCl, Ag and soot.

Further, the particle generation system includes:
a pneumatic atomization generator, a diffusion dryer, an evaporation condensation generator and a soot generator which are all connected to the solenoid valve system;
the pneumatic atomization generator is connected to the diffusion dryer; and
the pneumatic atomization generator is used for atomizing suspension including polystyrene particles into micron-sized droplets; the diffusion dryer is used for evaporating the micron-sized droplets to obtain solid polystyrene aerosol samples; the evaporation condensation generator is used for generating NaCl and Ag solid aerosol samples; and the soot generator is used for generating soot aerosol samples.

Specifically, the particle generation system mainly includes the pneumatic atomization generator, the diffusion dryer, the evaporation condensation generator, and the soot generator. The pneumatic atomization generator is used for atomizing suspension including polystyrene particles into micron-sized droplets, and the aqueous phase of the droplet is evaporated under the action of the diffusion dryer, thereby obtaining the solid polystyrene aerosol samples. The evaporation condensation generator is used for generating the NaCl and Ag solid aerosol samples. The soot generator is used for generating the soot aerosol samples. A computer is used to switch the solenoid valves 1-6, allowing different types of aerosol to enter the subsequent systems, i.e., particle classification and growth, particle mixing, and measurement system. For example, by opening solenoid valves 4 and 7 and closing solenoid valves 3, 5 and 6, only polystyrene aerosol can enter the subsequent systems, while by opening solenoid valves 1 and 2 at the same time, other aerosol samples are vented after filtration. By opening solenoid valves 5 and 7, and closing solenoid valves 2, 4, 6, only NaCl (or Ag) aerosol can enter the subsequent systems, while opening solenoid valves 1 and 3, other aerosol samples are vented after filtration. By opening solenoid valves 6 and 7, and closing solenoid valves 1, 4, 5, only soot aerosol can enter the subsequent systems, while opening solenoid valves 2 and 3, other aerosol samples are vented after filtration. In evaporation condensation generation technology, powder samples of NaCl or Ag are placed in a heating furnace, and the particle size and concentration of the aerosol samples are adjusted by adjusting the heating temperature, clean air flow, etc., and NaCl or Ag aerosol samples with a particle size of (10-120) nm and a concentration of not less than 10⁸/cm³ are produced. In soot generation technology, the particle size and concentration of soot aerosol samples are regulated by adjusting the flow rate of combustion gas, combustion-supporting gas, etc. By adjusting the opening and closing of the solenoid valve 7 and the flow rate, the aerosol samples can flow quantitatively into the neutralizer, while the excess aerosol samples are vented after filtration. In pneumatic atomization generation technology, by controlling the clean air pressure and selecting polystyrene samples with different particle sizes, the particle size and concentration of aerosol samples can be adjusted, and the dried polystyrene aerosol samples can be obtained after passing through a diffusion dryer.

Further, the particle growth system includes:
an aerosol neutralizer, a first DEMC, a second DEMC, a third DEMC, a CPC, a T-valve, a temperature controller, and a particle grower;
the aerosol neutralizer is connected to the solenoid valve system, the aerosol neutralizer is connected to the first DEMC, the DEMC and the third DEMC, the CPC is connected to the second DEMC, the third DEMC and the T-shaped valve, the DEMC is connected to the T-shaped valve, and the particle grower is connected to the T-shaped valve and the temperature controller; and
the aerosol neutralizer is used for neutralizing the charges of different types of aerosol samples to obtain aerosol samples in an overall electrically neutral state; the first DEMC is used for classifying the aerosol samples to obtain monodisperse aerosol samples; the DEMC is used for classifying the aerosol samples to obtain doubly-charged-particle aerosol samples; the third DEMC is used for classifying the aerosol samples to obtain triply-charged-particle aerosol samples; the CPC is used for measuring concentrations of the monodisperse aerosol samples, the doubly-charged-particle aerosol samples and the triply-charged-particle aerosol samples; the T-shaped valve is used for controlling the monodisperse aerosol samples to enter a particle measurement system; the temperature controller is used for controlling the temperature of the particle grower; and the particle grower is used for controlling the growth speed of the monodisperse aerosol samples according to the temperature.

Specifically, the particle classification and growth system (particle growth system) mainly includes the aerosol neutralizer, the DEMC, the particle grower, and the controller. The aerosol neutralizer is used for neutralizing the charges of the aerosol samples, thereby obtaining aerosol samples in an overall electrically neutral state. The DEMC 1 (the first DEMC) is used for classifying samples according to the difference in electrical mobility of charged particles in an electric field to obtain the monodisperse aerosol samples. The DEMC 2 (the second DEMC) and 3 (the third DEMC) are used by voltage setting for classifying doubly-charged-particle and triply-charged-particle aerosol samples. The particle grower includes a saturation chamber and a condensation chamber. When the monodisperse aerosol samples pass through the saturation chamber and subsequently enters the condensation chamber with a large amount of saturated working medium steam, due to the decrease in temperature, the saturated steam will condense on the surface of the monodisperse aerosol samples, thereby causing the size increase of solid particles. Since the selected working medium (e.g., carnauba hard wax) is solid at room temperature, the grown solid particles are solid particles. By adjusting temperatures of the condensation chamber and the saturation chamber, the controllable growth of the aerosol samples is realized.

Further, the particle measurement system includes:
a pressure gauge, a mixing and sampling tube, a particle size spectrometer, an aerosol electrometer, a PN analyzer, a constant flow pump, a first mass flowmeter and a second mass flowmeter;
one end of the mixing and sampling tube is connected to the T-shaped valve and the particle grower, the pressure gauge is mounted at a first preset position on the front end of the mixing and sampling tube, and the particle size spectrometer is arranged at a second preset position on the front end of the mixing and sampling tube; the particle number measuring instrument is arranged at a first preset position on a rear end of the mixing and sampling tube, and the aerosol electrometer is arranged at a second preset position on the rear end of the mixing and sampling tube; and the first mass flowmeter and the second mass flowmeter are connected to the constant flow pump, the second mass flowmeter is connected to the other end of the mixing and sampling tube, and the first mass flowmeter is connected to the aerosol electrometer; and
the mixing and sampling tube is used for mixing the monodisperse aerosol samples, the doubly-charged-particle aerosol samples and the triply-charged-particle aerosol samples; the particle size spectrometer is used for real-time monitoring the particle size distribution of aerosol samples; the pressure gauge is used for monitoring the aerosol pressure in the mixing and sampling tube; the aerosol electrometer is used for measuring the electrical current and particle concentration of charged particles; the constant flow pump is used for extracting aerosol samples from the mixing and sampling tube and the aerosol electrometer; the first mass flowmeter is used for controlling the measured flow of the aerosol electrometer; and the second mass flowmeter is used for controlling the aerosol flow in the mixing and sampling tube.

Specifically, the particle mixing and measurement system mainly includes a mixing and sampling tube, a particle size spectrometer, an aerosol electrometer, a PN analyzer of particles to be corrected, a constant flow pump, and a mass flow controller. The mixing and sampling tube is used for diluting and mixing the generated aerosol samples. The front section of the mixing and sampling tube is a turbulent mixing area of particle samples and diluted gases, and the rear end is the sampling area, enabling simultaneous isokinetic sampling and comparative measurements for six instruments. The particle size spectrometer is used for monitoring the particle size distribution of the aerosol samples in real time, and a pressure gauge is used for monitoring the pressure in the mixing and sampling tube, thereby correcting the sampling flow of the aerosol electrometer and the PN analyzer. The aerosol electrometer is a standard measuring instrument, and its measurement flow rate is continuously adjustable in the range of (0.3-5) L/min through mass flowmeters and the constant flow pump. It can output two measurement results: electrical current and PN concentration. The electrical current measurement results are corrected by the built-in calibration curve, while the PN concentration measurement results are corrected by multi-charge and flow rate. The PN analyzer to be calibrated is an instrument used for measuring the particle number from vehicle emission.

The present example also provides a traceable calibration method of PN analyzers for vehicle emission, including the following steps.

The aerosol electrometer and a PN analyzer are turned on, and the flow rate is adjusted and kept consistent.

The first DEMC, the second DEMC and the third DEMC are set to correspond to a first preset particle size, a second preset particle size, a third preset particle size, a first preset voltage size, a second preset voltage size and a third preset voltage size, and a sheath gas flow rate and an aerosol flow rate are kept consistent.

The opening and closing of corresponding solenoid valves in the solenoid valve system is used for controlling solid aerosol samples, i.e., polystyrene, NaCl, Ag and soot obtained by pneumatic atomization generator, evaporation condensation generator and soot generator, then, the aerosol samples were transmitted to the first DEMC, the second DEMC and the third DEMC to obtain aerosol samples with different charge states.

The CPC is used to measure concentrations of the aerosol samples with different charge states, followed by fractions are obtained according to the concentrations, and the correction of the measurement result of the aerosol electrometer is realized.

Aerosol samples through the first DEMC enter the mixing and sampling tube by shifting the T-shaped valve, and the flow and pressure of the mixing and sampling tube are controlled using the second mass flowmeter and dilution air.

Particle counting efficiencies of different types of aerosol samples are obtained according to the measured values, average values of the aerosol electrometer and the PN analyzer.

Specifically, the calibration method of the particle counting efficiency of the PN analyzer includes the following steps (taking the counting efficiencies for 100 nm and 200 nm particles as examples).

In step 1: the aerosol electrometer and PN analyzer are turned on, and a measured flow is adjusted to be consistent, and stable for at least 2 h.

In step 2: the classification particle diameter of the DEMC 1 is set to 100 nm (the corresponding voltage is U), and the voltages of the DEMCs 2 and 3 are set to 2 U and 3 U, where, the corresponding particle diameters are 146 nm and 195 nm; in the cases, the sheath gas flow rate and aerosol flow rate of the DEMCs 1, 2 and 3 are to be consistent.

In step 3, the solenoid valve 2 is opened, and the NaCl powder is placed in the evaporation condensation generator. The temperature of a tube furnace of the evaporation condensation generator is set to (700±20° C.), and the carrier gas flow rate is set to (2-10) L/min.

In step 4, the solenoid valve 3 is opened, 100 nm polystyrene suspension is dropped into a liquid storage bottle of the pneumatic atomization generator, compressed air is introduced into the pneumatic atomization generator, and the atomization pressure is adjusted.

In step 5: the solenoid valve 1 is opened, combustion gas (propane) and combustion-supporting gas (air) are introduced into the soot generator, and the electronic igniter is turned on to make the combustion gas burn stably and quantitatively produce soot aerosol particles.

In step 6: the solenoid valve 2 is closed, the solenoid valve 5 is opened, the NaCl aerosol samples enter the DEMCs 1, 2 and 3 after passing through an aerosol splitter, and produces monodisperse aerosol samples with positive charges.

In step 7: the particle concentrations of the DEMCs 1, 2, and 3 are measured using a CPC, denoted as C_{N}(d₁), C_{N}(d₂) and C_{N}(d₃). The computer calculates the fraction of particles carrying 1, 2 and 3 charges in the 100 nm aerosol samples according to the following formula.

$${C}_{1} ( {d}_{2} ) = \frac{{C}_{N} ( {d}_{2} )}{{η}_{C}} \qquad ( 1 )$$

$${C}_{1} ( {d}_{3} ) = \frac{{C}_{N} ( {d}_{3} )}{{η}_{C}} \qquad ( 2 )$$

$${C}_{2} ( {U}_{1} ) = {C}_{2} ( {d}_{2} ) = {C}_{1} ( {d}_{2} ) \times \frac{{f}_{2} ( {d}_{2} )}{{f}_{1} ( {d}_{2} )} \qquad ( 3 )$$

$${C}_{3} ( {U}_{1} ) = {C}_{3} ( {d}_{3} ) = {C}_{1} ( {d}_{3} ) \times \frac{{f}_{3} ( {d}_{3} )}{{f}_{1} ( {d}_{3} )} \qquad ( 4 )$$

$${φ}_{p} = \frac{{C}_{p} ( {U}_{1} )}{{C}_{N} ( {d}_{1} ) / {η}_{C}} \times 100 \% \qquad ( 5 )$$

where:
η_{C} is a particle counting efficiency of the CPC, which is a dimensionless quantity; C₁(d₂) and C₂(d₂) are PN concentration values of 146 nm monodisperse aerosol samples with surfaces carrying one and two positive charges, respectively, in particles/cm³; f₁(d₂) and f₂(d₂) are the charging probability for 146 nm particles carrying 1 and 2 positively charges, C₁(d₃) and C₃(d₃) are PN concentration values of 195 nm monodisperse aerosol samples with surfaces carrying 1 and 3 positive charges, in particles/cm³; and f₁(d₃) and f₃(d₃) are charging probability for 146 nm particles carrying 1 and 3 positive charges.

In step 8: the T-shaped valve is switched, allowing the aerosol sample classified by the DEMC 1 enters the mixing and sampling tube.

In step 9: the flow rate of the mass flow controller is controlled in a range of (1-5) L/min, and the dilution air is introduced to make the pressure value within (−30-0) kPa.

In step 10: the measured values and average values of the aerosol electrometer and the PN analyzer are recorded, and the particle counting efficiency of the PN analyzer for the 100 nm polystyrene samples is calculated according to the formula (6).

$${η}_{PN} = \frac{{\overline{C}}_{PN}}{{\overline{C}}_{F}} \times {η}_{F} \times {\sum }_{p = 1}^{n} ( {φ}_{p} \times p ) \times 100 \% \qquad ( 6 )$$

where η_{PN} is a particle counting efficiency of the PN analyzer to be calibrated; C̅_{PN} is an average of measurement results of the PN analyzer to be calibrated, in particles/cm³; C̅_{F} is an average of measurement results of the aerosol electrometer, in particles/cm³; η_{F} is a particle counting efficiency of the aerosol electrometer, which is a dimensionless quantity; φₚ is a fraction of particles carrying p charges in aerosol particles, which is a dimensionless quantity; and p is the number of positive or negative charges carried by particles, which is a dimensionless quantity.

In step 11: the solenoid valves 3 and 5 are closed, the solenoid valves 2 and 4 are opened, the polystyrene aerosol samples enter the DEMCs 1, 2 and 3 after passing through the aerosol splitter, and monodisperse aerosol samples with positive charges are produced. After that, steps 7 to 10 are repeated to obtain the particle counting efficiency of the PN analyzer for the 100 nm polystyrene samples.

In step 12: the solenoid valves 1 and 5 are closed, the solenoid valves 2 and 6 are opened, the soot aerosol samples enter the DEMCs 1, 2 and 3 after passing through the aerosol splitter, and monodisperse aerosol samples with positive charges are produced. After that, steps 7 to 10 are repeated to obtain the particle counting efficiency of the PN analyzer for the 100 nm soot samples.

In step 13: when calibrating the counting efficiency of PN analyzer for 200 nm aerosol samples, steps 1 to 7 are first repeated, the T-shaped valve is switched, the aerosol samples classified by the DEMC 1 enter the particle grower, the temperature of the saturation chamber and the condensation chamber in the particle grower are adjusted to be within ranges of (100-300° C.) and (10-50° C.), and the working medium in the grower condenses and grows to 200 nm on the particle surface. The particle size of the grown particle samples is monitored using the particle size spectrometer. Steps 9-10 are repeated to obtain the particle counting efficiency of the PN analyzer for the 200 nm samples.

Each example in this specification is described in a progressive manner, and each example focuses on the differences from other examples. The same and similar parts of each example can be referred to each other.

While the principles and embodiments of the present disclosure have been described herein using specific examples, the description of the above examples is intended only to aid in the understanding of the method and core idea of the present disclosure. Meanwhile, for those skilled in the art, according to the idea of the present disclosure, changes will occupy a place in the specific embodiments and application ranges. In view of the above, this specification is not to be construed as limiting the present disclosure.

1. A traceable calibration device of particle number (PN) analyzers for vehicle emission, comprising:
a particle generation system, a solenoid valve system, a particle growth system and a particle measurement system connected in sequence, wherein
the particle generation system is used for generating different types of aerosol samples; the solenoid valve system is used for controlling various aerosol samples to quantitatively enter the particle growth system; the particle growth system is used for classifying different types of aerosol samples to obtain monodisperse aerosol samples, doubly-charged-particle aerosol samples and triply-charged-particle aerosol samples, determining concentrations of the three aerosol samples, and obtaining grown particle samples by controlling the growth speed of the monodisperse aerosol samples through temperature adjustment; the particle measurement system is used for measuring the grown particle samples to obtain output electrical current, PN concentration; and the solenoid valve system is configured with seven electromagnetic valves, and the different types of solid aerosol samples comprise polystyrene, NaCl, silver (Ag) and soot.

2. The traceable calibration device of PN analyzers for vehicle emission according to claim 1, wherein the particle generation system comprises:
a pneumatic atomization generator, a diffusion dryer, an evaporation condensation generator and a soot generator, which are all connected to the solenoid valve system;
the pneumatic atomization generator is connected to the diffusion dryer; and
the pneumatic atomization generator is used for atomizing suspension comprising polystyrene particles into micron-sized droplets; the diffusion dryer is used for evaporating the micron-sized droplets to obtain solid polystyrene aerosol samples; the evaporation condensation generator is used for generating NaCl or Ag solid aerosol samples; and the soot generator is used for generating soot aerosol samples.

3. The traceable calibration device of PN analyzers for vehicle emission according to claim 2, wherein the particle growth system comprises:
an aerosol neutralizer, a first differential electrical mobility classifier (DEMC), a second DEMC, a third DEMC, a condensation particle counter (CPC), a T-valve, a temperature controller, and a particle grower;
the aerosol neutralizer is connected to the solenoid valve system, the aerosol neutralizer is connected to the first DEMC, the second DEMC and the third DEMC, the CPC is connected to the second DEMC, the third DEMC and the T-shaped valve, the first DEMC is connected to the T-shaped valve, and the particle grower is connected to the T-shaped valve and the temperature controller; and
the aerosol neutralizer is used for neutralizing the charges of different types of aerosol samples, and obtaining aerosol samples in an overall electrically neutral state; the first DEMC is used for classifying the aerosol samples to obtain monodisperse aerosol samples; the second DEMC is used for classifying the aerosol samples to obtain doubly-charged-particle aerosol samples; the third DEMC is used for classifying the aerosol sample to obtain triply-charged-particle aerosol samples; the CPC is used for measuring concentrations of the monodisperse aerosol samples, the doubly-charged-particle aerosol samples and the triply-charged-particle aerosol samples; the T-shaped valve is used for controlling the monodisperse aerosol samples to enter a particle measurement system; the temperature controller is used for controlling the temperature of the particle grower; and the particle grower is used for controlling the growth speed of the monodisperse aerosol samples according to the temperature.

4. The traceable calibration device of PN analyzers for vehicle emission according to claim 3, wherein the particle measurement system comprises:
a pressure gauge, a mixing and sampling tube, a particle size spectrometer, an aerosol electrometer, a PN analyzer, a constant flow pump, a first mass flowmeter and a second mass flowmeter;
one end of the mixing and sampling tube is connected to the T-shaped valve and the particle grower, the pressure gauge is mounted at a first preset position on the front end of the mixing and sampling tube, and the particle size spectrometer is arranged at a second preset position on the front end of the mixing and sampling tube; the particle number measuring instrument is arranged at a first preset position on the rear end of the mixing and sampling tube, and the aerosol electrometer is arranged at a second preset position on the rear end of the mixing and sampling tube; and the first mass flowmeter and the second mass flowmeter are connected to the constant flow pump, the second mass flowmeter is connected to the other end of the mixing and sampling tube, and the first mass flowmeter is connected to the aerosol electrometer; and
the mixing and sampling tube is used for mixing the monodisperse aerosol samples, the doubly-charged-particle aerosol samples and the triply-charged-particle aerosol samples; the particle size spectrometer is used for real-time monitoring the particle size distribution of aerosol samples; the pressure gauge is used for monitoring the aerosol pressure in the mixing and sampling tube; the aerosol electrometer is used for measuring the electrical current and particle concentration of charged particles; the constant flow pump is used for extracting aerosol samples in the mixing and sampling tube and the aerosol electrometer; the first mass flowmeter is used for controlling the measured flow of the aerosol electrometer; and the second mass flowmeter is used for controlling the aerosol flow in the mixing and sampling tube.

5. A traceable calibration method of PN analyzers for vehicle emission, applied to the device according to claim 4, comprising the steps of:
turning on the aerosol electrometer and the PN analyzer, adjusting flow rates, and keeping the flow rates consistent;
setting the first DEMC, the second DEMC and the third DEMC to correspond to a first preset particle size, a second preset particle size, a third preset particle size, a first preset voltage size, a second preset voltage size and a third preset voltage size, and keeping a sheath gas flow rate and an aerosol flow rate consistent;
controlling the opening and closing of corresponding solenoid valves in the solenoid valve system, obtaining different solid aerosol samples, i.e., polystyrene, NaCl, Ag and soot by using the pneumatic atomization generator, the evaporation condensation generator, and the soot generator, and transmitting the samples to the first DEMC, the second DEMC and the third DEMC are used to obtain aerosol samples with different charge states;
using the CPC to measure concentrations of the aerosol samples with different charge states and obtain fractions according to the concentrations, and realizing the correction of the measurement result of the aerosol electrometer;
causing aerosol samples through the first DEMC to enter the mixing and sampling tube according to the T-shaped valve, and controlling the flow and pressure of the mixing and sampling tube using the second mass flowmeter and dilution air; and
obtaining particle counting efficiencies of different types of aerosol samples according to the measured values, average values of the aerosol electrometer and the PN analyzer.

6. The traceable calibration method of PN analyzers for vehicle emission according to claim 5, wherein an expression of the efficiency formula is:
$${η}_{PN} = \frac{{\overline{C}}_{PN}}{{\overline{C}}_{F}} \times {η}_{F} \times {\sum }_{p = 1}^{n} ( {φ}_{p} \times p ) \times 100 \%$$
where η_{PN} is a particle counting efficiency of the PN analyzer to be calibrated; C̅_{PN} is an average of measurement results of the PN analyzer to be calibrated; C̅_{F} is an average of measurement results of the aerosol electrometer; η_{F} is a particle counting efficiency of the aerosol electrometer; φₚ is a fraction of particles carrying p charges in aerosol particles; p is the number of positive and negative charges carried by particles, and n is a natural number.
